(19) United States
(12) Patent Application Publication
(10) Pub. No.: US 2015/0342484 A1
(43) Pub. Date: Dec. 3, 2015

(54) EVALUATION BY OCTAVE ANALYSIS

(71) Applicant: Burkhard Karl-Heinz Gunther FRANZ, Melbourne (AU)

(72) Inventor: Burkhard Karl-Heinz Gunther FRANZ, Melbourne (AU)

(73) Assignee: BURKHARD FRANZ PTY LTD, Melbourne (AU)

(21) Appl. No.: 14/345,254

(22) PCT Filed: Sep. 14, 2012
(86) PCT No.: PCT/AU2012/001092

§ 371 Date: Jun. 19, 2015

(30) Foreign Application Priority Data
Sep. 16, 2011 (AU) 2011903812

(51) Int. Cl. A61B 5/04; A61N 1/05; A61B 5/12

(52) U.S. Cl. CPC A61B 5/04001; A61B 5/125; A61N 1/0541

(57) ABSTRACT

A method of examining a bodily function. A first set of frequency characteristics and amplitude characteristics of a first electrical signal deriving from rhythmic activity of a nerve in a living body is detected at a first condition. The nerve is stimulated to a second condition and a second set of frequency characteristics and amplitude characteristics of a second electrical signal deriving from the rhythmic activity of said nerve is detected. The fundamental frequency present in the frequency characteristics is identified. From the fundamental frequency the frequencies corresponding to a plurality of octaves of said fundamental frequency are calculated. The amplitude of said electrical signals at said octaves is identified. For said first signal a first amplitude ratio is calculated being the ratio of the amplitude difference between a first pair of said octaves, and the amplitude difference between a second pair of said octaves. For said second signal a second amplitude ratio is calculated being the ratio of the amplitude difference between said first pair of octaves, and the amplitude difference between said second pair of octaves. An octave ratio is calculated being the ratio of said first amplitude ratio to said second amplitude ratio. The octave ratio is used as a measure of said bodily function.

### FIELD OF THE INVENTION

This invention concerns the detection and use of electrical signals produced within a living body in order to provide an indication of the condition of the body. The invention has particular application in the field of electrovestibulography for the purpose of diagnosing disfunctions in humans.

### BACKGROUND TO THE INVENTION

Averaging is an important and well recognised technique to assess cochlear function (1). It has an important role to play in the differential diagnosis of inner ear disorders such as in Ménière's disease. A similar development to assess vestibular function has been missing. This is surprising since the recording electrode in electrocochleography placed close to the round window would also be suited to record vestibular responses.

There have been previous attempts to average peripheral vestibular responses employing electrovestibulography recording techniques (2). In this technique harmonics are analysed. Averaging is performed by adding responses that are phase locked to a harmonic of the vestibular nerve response. A disadvantage of this technique is the need for training, i.e. to learn how to interpret the developing real time signal on the monitor and save it at the right moment. This disadvantage has made this technique less objective.

Clinical tests can be employed to assess vestibular function. Popular are the Romberg test, Unterberger stepping test, Dix-Hallpike manoeuvre and Halmagyi impulse test (3-6). Also electrophysiological tests are available to assess vestibular function. Nystagmography is employed to assess semicircular canal function, vestibular evoked myogenic potentials to assess saccular function and cranio-corpography to assess the vestibulo-spinal axis in general (7-9). A disadvantage of the former two is that one sensory organ is used to assess another sensory organ.

The latter assesses the vestibular system as a whole without specific information on individual units of the peripheral vestibular organ.

The present invention has been developed to address the abovementioned limitations but it is considered that the invention in its general form can be utilised not just for electrovestibulography but also for diagnostic purposes based on the detection and analysis of electrical signals deriving from nerve function in other parts of the body.

### SUMMARY OF THE INVENTION

Accordingly, in one aspect the invention provides a method of examining a bodily function comprising:
detecting a first set of frequency and amplitude characteristics of a first electrical signal deriving from rhythmic activity of a nerve in a living body at a first condition;
stimulating said nerve and detecting a second set of frequency and amplitude characteristics of a second electrical signal deriving from the rhythmic activity of said nerve while stimulated;
identifying the fundamental frequency present in said frequency characteristics;
calculating from said fundamental frequency the frequencies corresponding to a plurality of octaves of said fundamental frequency;
identifying the amplitude of said electrical signals at said octaves;
calculating for said first signal a first amplitude ratio being the ratio of:
   the amplitude difference between a first pair of said octaves and
   the amplitude difference between a second pair of said octaves;
calculating for said second signal a second amplitude ratio being the ratio of:
   the amplitude difference between said first pair of octaves and
   the amplitude difference between said second pair of octaves;
calculating an octave ratio being the ratio of said first amplitude ratio to said second amplitude ratio; and
using said octave ratio as a measure of said bodily function.

In another aspect the invention provides a method of diagnosing a bodily dysfunction, said method comprising:
detecting a first set of frequency and amplitude characteristics of a first electrical signal deriving from rhythmic activity of the peripheral vestibular nerve in a living body at a first condition;
stimulating said nerve and detecting a second set of frequency and amplitude characteristics of a second electrical signal deriving from the rhythmic activity of said nerve while stimulated;
identifying the fundamental frequency present in said frequency characteristics;
calculating from said fundamental frequency the frequencies corresponding to a plurality of octaves of said fundamental frequency;
identifying the amplitude of said electrical signals at said octaves;
calculating for said first signal a first amplitude ratio being the ratio of:
   the amplitude difference between a first pair of said octaves and
   the amplitude difference between a second pair of said octaves;
calculating for said second signal a second amplitude ratio being the ratio of:
   the amplitude difference between said first pair of octaves and
   the amplitude difference between said second pair of octaves;
calculating an octave ratio being the ratio of said first amplitude ratio to said second amplitude ratio; and
using said octave ratio as a measure of said bodily dysfunction.

Preferably the octaves are selected from octaves 1 to 7, and more preferably from octaves 2 to 4.

Preferably said first amplitude ratio is the ratio of the amplitude of octave 2 to the amplitude of octave 3, and said second amplitude ratio is the ratio of the amplitude of octave 2 to the amplitude of octave 4.

Preferably said first amplitude ratio is the ratio of:
the amplitude difference between octave 2 and octave 3 of said fundamental frequency, and
the amplitude difference between octave 2 and octave 4 of said fundamental frequency.

### BRIEF DESCRIPTION OF THE FIGURES

In order that the invention may be more fully understood there will now be described, by way of example only, preferred embodiments and other elements of the invention with reference to the accompanying Figures where:

FIG. 1 is a frequency spectrogram showing harmonics in the lower frequency range of a vestibular nerve response.

FIG. 2 is a diagram showing octave ratios in roll and in pitch in the left ear of a normal subject.

FIG. 3 is a diagram showing octave ratios in roll and in pitch in the right ear of the same subject as in FIG. 2.

FIG. 4 is a diagram showing octave ratios following cold and warm stimulation in both ears in the same subject as in FIGS. 2 and 3.

FIG. 5 is a diagram showing octave ratios in roll and in pitch in a patient with benign paroxysmal positioning vertigo.

FIG. 6 is a diagram showing octave ratios following cold and warm stimulation in a patient with vestibular neuritis in the right ear.

FIG. 7 is a diagram showing octave ratios in roll and in pitch in a patient with labyrinthitis in the left ear.

### DESCRIPTION OF EXAMPLES OF THE INVENTION

The endogenous activities of the peripheral vestibular organ were investigated employing an electrode configuration like in electrocochleography. Preliminary analyses of raw data and clinical tests confirmed rhythmic activity of the vestibular nerve, but above all could demonstrate a sequence of octaves in the lower frequency response with fundamental frequencies around 23 Hz.

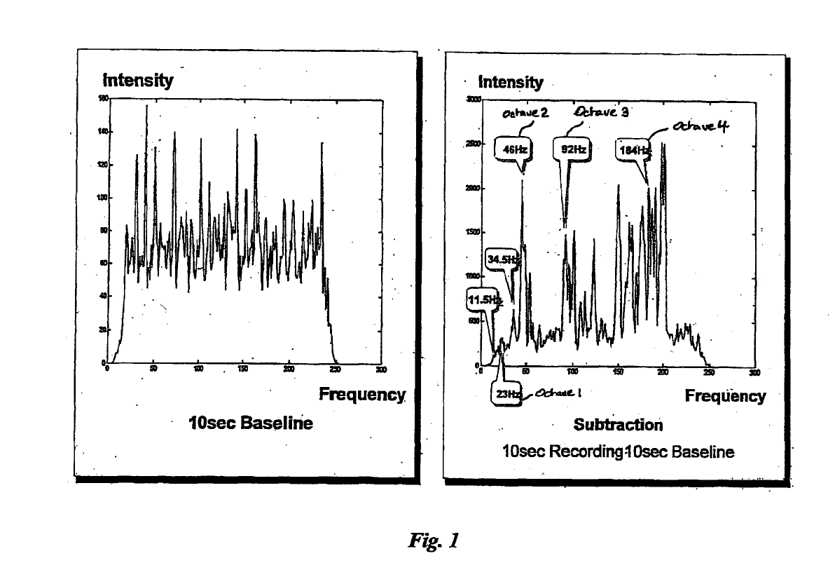

In FIG. 1 a frequency spectrogram is showing harmonics in the lower frequency range of a vestibular nerve response. The fundamental frequency of the harmonics is 23 Hz. Amplitudes (μV) of O2, O3 and O4 are used to calculate the octave ratio.

Pilot studies suggested that the most significant parameters were the amplitudes measured in microvolts of octave 2, 3 and 4 and their relationship to each other. This relationship can be expressed in a ratio calculated by the amplitude difference of octaves 2 to 3 and octaves 2 to 4. The Jongkees formula then allows calculating a significant or insignificant vestibular deficit (10).

It has been found that the amplitude of octaves 2, 3 and 4 change following stimulation, that their relationship to each other also changes, and that these changes are significant in patients with vestibular dysfunction.

The electrode layout in the electrovestibulography was the same as in electrocochleography. The recording electrode was placed on the ear drum, the reference electrode on the mastoid plane and the ground electrode was placed on the vertex.

Raw data were collected without a predetermined starting point over a period of 21 seconds with a collection rate of only 1000 points per second (PSYLAB Precision Instruments, London NW32 TB). A further simplification of data was introduced by averaging every three consecutive recordings reducing the collection points to 333 per second. From these data the amplitudes (μV) of seven octaves were calculated at fundamental frequencies ranging from 21 Hz to 25 Hz considering every decimal point. Amplitude difference of octave 2 to 3 was divided by amplitude difference 2 to 4 to determine the octave ratio.

The otolithic organs were stimulated by tilting the head in roll and in pitch. It was reasonably assumed that ipsilateral and contralateral head tilt, i.e. roll would mainly stimulate the utriculus, whereas forward and backward head tilt, i.e. pitch would mainly stimulate the sacculus. Recording started within two seconds following stimulation.

The lateral semicircular canal was stimulated with cold and warm air at 27° and 47° Celsius blown into the ear canal for 45 seconds while the electrode remained in situ (Caloric Otometer, Airmatic, Hortmann, Germany). Recording started within two seconds following termination of the stimulus.

In assessing roll, ipsilateral head tilt was compared with contralateral head tilt and ipsilateral head tilt being the reference. It was not referenced to a baseline response as repeated baseline measurements were very unstable. The same principle applied in assessing pitch where forward head tilt was compared with backward head tilt, and forward head tilt being the reference.

Cold and warm stimuli of the lateral semicircular canal were referenced to a baseline recording. Here repeated baseline measurements were very stable with the subject lying comfortably on the bench with the head elevated by 30° and practically motionless.

The first step was to determine the reference ratio. It was found by calculating the smallest 2-3/2-4 octave ratio at a frequency of raw data between 21 Hz a 25 Hz. Once the reference ratio was established the octave ratio from another stimulus was compared to the reference ratio at exactly the same frequency of the reference ratio.

The Jongkees formula was used to determine whether there was a significant deficit in the response of one vestibular organ. The notation deficit rather than the notation preponderance was preferred as better suited in this concept.

A study was undertaken with 97 patients with cochleo-vestibular disorders: benign paroxysmal positioning vertigo (14), vestibular neuritis (6), labyrinthitis (6), Ménière's disease (6), migraine (30), general viral infections (25), acoustic neuroma (2), Chiari malformation (2), and diabetes (12). Diagnoses relied on the history, clinical examination and conventional otoneurological assessments without octave analysis. These were in particular: pure tone audiometry, tympanometry, brainstem audiometry, electrocochleography, vestibular evoked myogenic potentials, electronystagmography, and presence of spontaneous or evoked nystagmus, Romberg test, Unterberger stepping test, Halmagyi impulse test and Dix-Hallpike manoeuvre, and in some cases MRI scans of the brain.

For statistical analysis the student t-test (α<0.05) was used.

Normal data were obtained from five subjects without cochleo-vestibular symptoms. They had normal hearing levels, normal brainstem audiograms and normal electrocochleograms. There were two females and three males. Their ages ranged from 33 to 69 years (average 41 years).

Table 1 shows octave analysis in roll and in pitch for those normal subjects. Normal responses are expected to be below 46% in roll and below 55% in pitch. In Table 2 cold and warm stimuli were referenced to the cold baseline recording with the patient lying on a bench and the head elevated by 30°. Normal responses are expected to be below 33%.

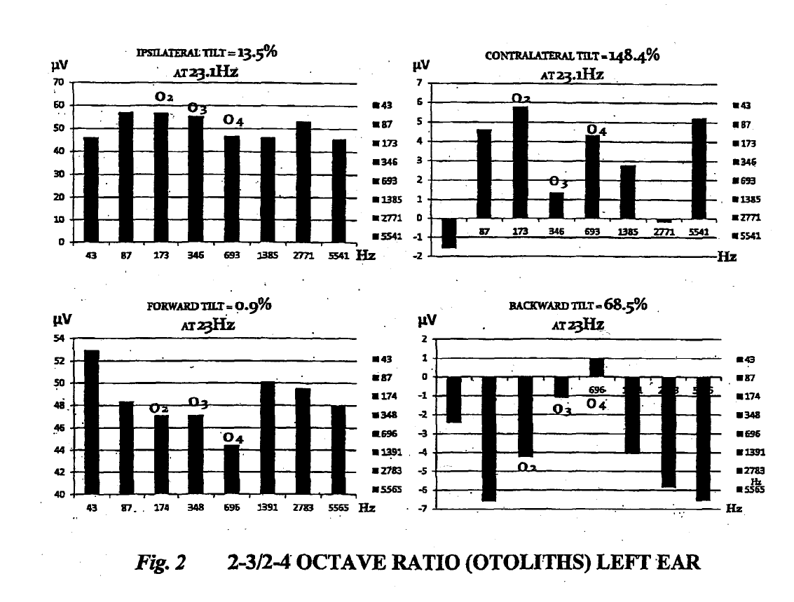

Normal responses in roll (utricular) were found to be below 46% using the Jongkees formula. Normal responses in pitch (saccular) were found below 55%, and normal responses in the calorics were found below 33%. FIGS. 2, 3 and 4 show the response of a subject without vestibulo-cochlear symptoms.

FIG. 2 is a diagram showing octave ratios in roll and in pitch in the left ear of a normal subject. The reference ratio in ipsilateral head tilt was 13.5% at a frequency of 23.1 Hz. In contralateral head tilt the ratio changed to 148.4% at exactly the same frequency of 23.1 Hz. Similarly the reference ratio in forward head tilt was 0.9% at a frequency of 23 Hz. In backward head tilt it changed to 68.5% at exactly the same frequency of 23 Hz.

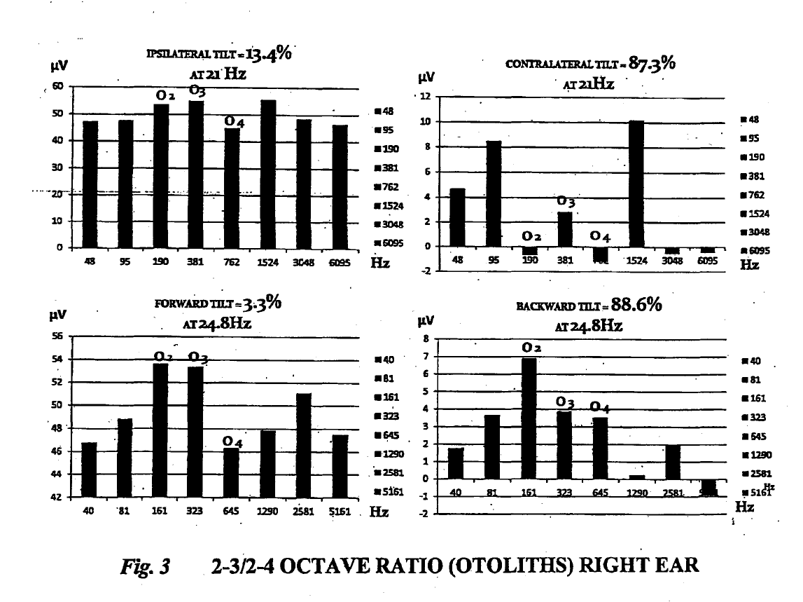

FIG. 3 is a diagram showing octave ratios in roll and in pitch in the right ear of the same subject as in FIG. 2. There is a dramatic change of octave ratios in contralateral and backward head tilt when compared to the reference ratios of forward and backward head tilts. Note that these measurements are at exactly the same respective frequencies. The result of the Jongkees formula revealed a deficit within normal limits: 23.7% roll deficit in the left ear and 13.6% pitch deficit in the right ear (N<46% and <55% respectively).

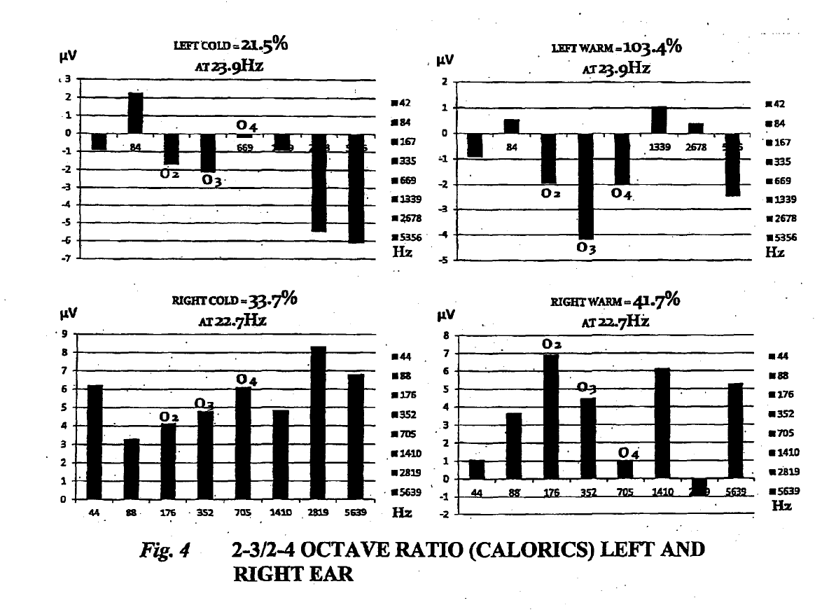

FIG. 4 is a diagram showing octave ratios following cold and warm stimulation in both ears in the same subject as in FIGS. 2 and 3. The smallest octave ratio in the baseline recording was found at 23.9 Hz in the left ear and at 22.7 Hz in the right ear. Following cold stimulation the ratio changed at exactly the same frequency to 21.5% in the left ear and to 33.7% in the right ear. Following warm stimulation the ratio at exactly the same frequency changed to 103.4% in the left ear and 41.7% in the right ear. The result of the Jongkees formula revealed a deficit within normal limits: 24.4% caloric deficit left ear (N<33%).

Patients with benign paroxysmal positioning vertigo always showed a significant deficit in the otolithic organ on the side of the affected ear. Typically the history, the Dix-Hallpike manoeuvre and Unterberger stepping test were supportive of the diagnosis.

In vestibular neuritis a significant deficit was always observed in the calorics on the side of the affected ear. Here the diagnosis was supported by the history, pure tone audiometry, brainstem audiometry, the Unterberger stepping test, Romberg test and the Halmagyi head impulse test.

In viral labyrinthitis and Ménière's disease a deficit was paradoxically always found in the otolithic organ of the opposite ear. The history and pure tone audiometry, brainstem audiometry and the electrocochleogram were supportive of the diagnosis.

In Chiari malformations, migraine, diabetes, degenerative disorders, genetic disorders and general viral infections both the otolithic organ and or lateral semicircular canal were found to have deficits. There was always a bilateral involvement with all kinds of patterns. Here often the Unterberger stepping test and Romberg tests were contradictive.

Two patients had an early Acoustic Neuroma where a vestibular deficit could not be detected although in one case the vestibular response was just within normal limits on the side of the affected ear. This patient complained of a long standing instability, but no vertigo. The hearing was normal in these two patients. It was the abnormal brainstem audiogram that led to the diagnosis. MRI scans revealed small intracanalicular tumours.

Some specific examples will now be described.

### 1. Benign Paroxysmal Positioning Vertigo.

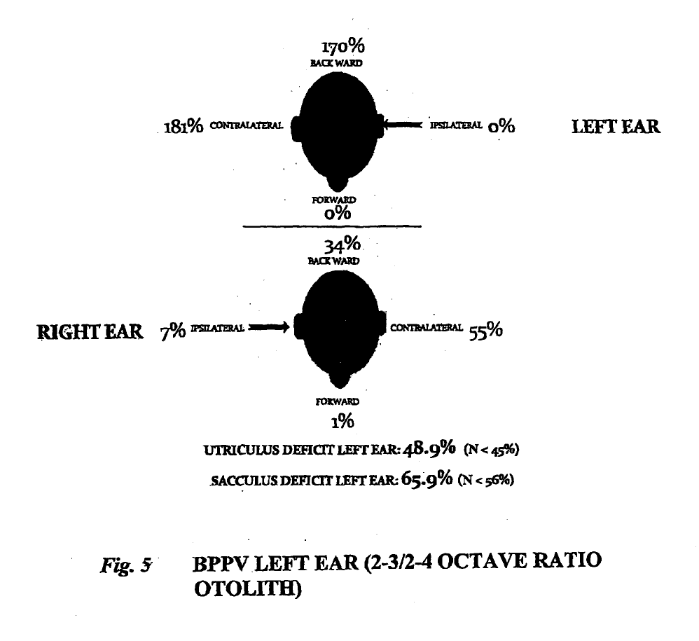

A 55 year old lady presented with a recent history of positioning vertigo. Turning in bed towards the left caused temporary vertigo over approximately 20 seconds. Hearing was normal. Brainstem audiometry and electrocochleography were unremarkable. The Dix-Hallpike manoeuvre was positive in the left ear. During the Romberg test the left arm was lowered, and during the Unterberger stepping test she turned to the left. As shown in FIG. 5, there was a significant roll and pitch deficit in the left ear: 48.5% roll deficit left ear and 65.9% pitch deficit in the left ear (N<46% and <55% respectively). The calorics were within normal limits.

### 2. Vestibular Neuritis Right Ear.

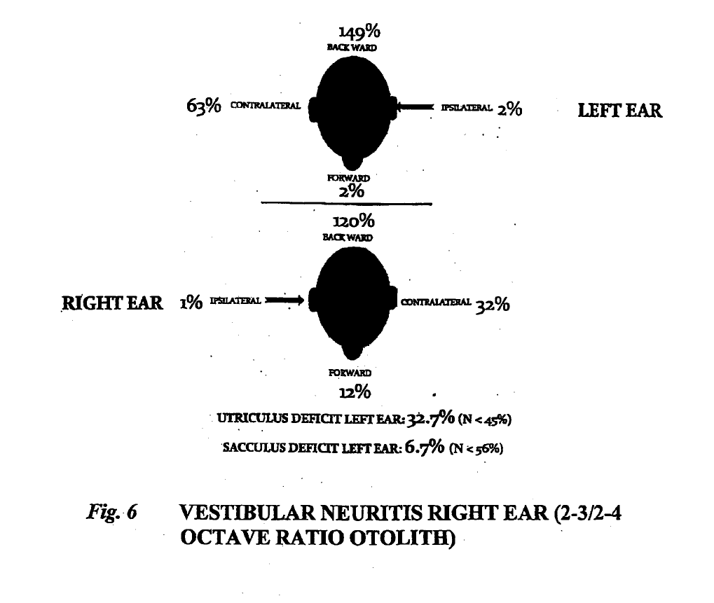

A 44 year old lady presented with a recent history of vertigo. She woke up one morning with severe vertigo and vomiting following a chest infection two weeks prior. The hearing was within normal limits. Brainstem audiometry and electrocochleography were normal. There was a temporary nystagmus beating into the left ear after head shaking. The Dix-Hallpike manoeuvre was clear. She could not perform the Unterberger stepping test. During the Romberg test she fell backwards to the right. The Halmagyi impulse test was positive for the right ear. There was no significant deficit detectable in roll or in pitch. However, as shown in FIG. 6, there was a significant deficit detected in the right ear: 48% caloric deficit right ear (N<33%).

### 3. Viral Labyrinthitis Left Ear.

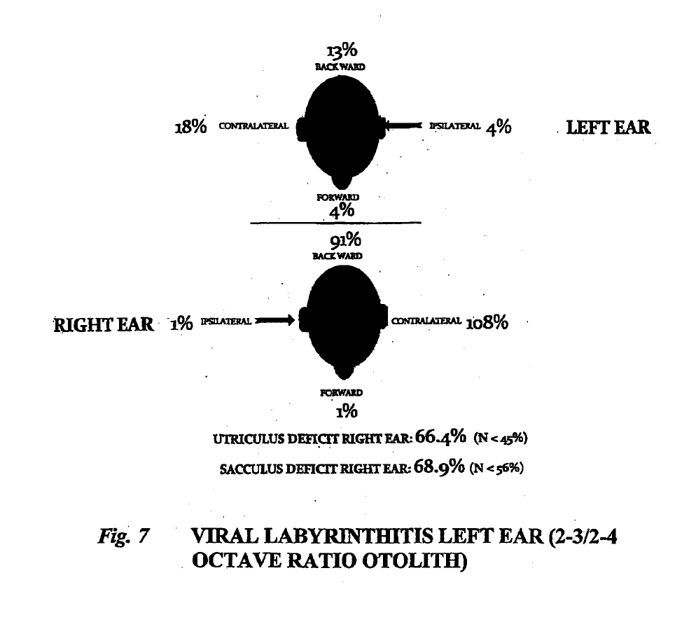

A 56 year old male presented with a recent history of vertigo and severe pain in the left ear following flu. There was a mild to moderate high frequency sensorineural hearing loss in both ears and significantly worse in the left ear. Brainstem audiometry was normal. The electrocochleogram revealed a hydrops in the left ear. The Dix-Hallpike manoeuvre was unremarkable. During the Romberg test he was unstable, and during the Unterberger stepping test he turned to the left. The Halmagyi impulse test was unremarkable. As shown in FIG. 7, octave ratios in roll and in pitch revealed a significant deficit, but in the non-affected right ear: 66.4% roll deficit in right ear and 68.9% pitch deficit in right ear (N<46% and <55% respectively). This apparent paradox response was also typically found in all cases of Ménière's disease.

Relatively few attempts have been made to examine the peripheral vestibular organ in the human and animal with evoked potentials (2, 11-13). These have not translated into clinical application. This is disappointing since the recording electrode placed close to the round window would be ideally placed to record vestibular potentials.

A major difficulty in applying electrovestibulography in the human with averaging techniques is the inability to effectively phase lock to a stimulus frequency. The period between consecutive stimuli is too long to obtain a sufficient number of sweeps for accurate averaging.

Instead of using averaging the present invention uses the endogenous activities of the vestibular organ. Vestibular neurons have been found to have inbuilt regular and irregular activities in squirrel monkeys (14). It has now been found that regular activities in the vestibular nerve response of the lateral semicircular canal and the otolithic organ can be found in the human as well. Harmonics in the vestibular nerve response were previously successfully employed in averaging. Instead of adding responses phase locked to a stimulus frequency, responses could be added that were phase locked to a harmonic of a response frequency (2).

Regular endogenous activity of the otolithic organ and lateral semicircular canal is demonstrated by the presence of octaves with fundamental frequencies ranging from 21 Hz to 25 Hz. The relationship of amplitudes measured in microvolts of octaves 2, 3 and 4 expressed in a ratio changes significantly in vestibular dysfunction. This new concept of electrovestibulography promises an interesting addition to the electrophysiological assessment of the inner ear and its differential diagnosis.

It has now been found in studies of the vestibular nerve observations that sequences of octaves occur with a fundamental frequency always hovering around 23 Hz. Octaves 1 to 7, and their relationship to each other have been identified as the most interesting. The size of the amplitudes of these octaves measured in microvolts invariably changed under various conditions, but individual octave voltages did not show statistically significant changes. The variation of voltages was too great. However, the ratio of octaves 2 to 3 and 2 to 4 revealed a significant change in subjects with vestibular dysfunction.

While nystagmography assesses lateral canal function it does it via the assessment of another sensory organ. This makes the test less specific. Similarly vestibular evoked myogenic potentials assess saccular function also by assessing the response of another organ, and it does not provide information on utricular function. The advantage of octave analysis is a direct assessment of vestibular function of the lateral semicircular canal and both otolithic organs, and it can easily follow electrocochleography as the electrode configuration is the same.

Patients with benign paroxysmal positioning vertigo, vestibular neuritis, labyrinthitis and Ménière's disease had a consistent and distinct ratio pattern. Here a vestibular deficit was found in one ear, usually on the side of the affected ear. Patients with migraine, Chiari malformation, genetic and degenerative disorders, general viral infections and diabetes showed a rather diffuse pattern. Here the vestibular organ of both ears showed a deficit, and the pattern was unpredictable. The involvement of both ears would suggest an underlying common cause.

Why, however, in Ménière's disease and labyrinthitis a vestibular deficit was detected in the opposite ear is not clear. It was also intriguing that the deficit in the contralateral ear contradicted the clinical Unterberger stepping test. An explanation could be that tilting the head will always stimulate both otolithic organs and the response could be influenced by the efferent system. One might speculate that the hydrops in one ear could cause this apparent paradox response. An alternative explanation would be recruitment. Recruitment renders the affected ear more sensitive to stimulation and could make the opposite ear only appear to have a deficit.

By way of summarizing the above description, octave amplitudes in the lower frequency response (fundamental frequency 21 Hz to 25 Hz) of the peripheral vestibular nerve were analysed using electrovestibulography recording techniques. Otolithic organs were stimulated by tilting the head in roll and in pitch. The lateral semicircular canal was stimulated with cold and warm air blown into the ear canal at 27° and 47° Celsius. The amplitudes measured in microvolts changed under various conditions, particularly the ratio calculated by the amplitude difference of octaves 2 to 3 and the amplitude difference of octaves 2 to 4. These changes were significant in patients with vestibular dysfunction. Patients with benign paroxysmal positioning vertigo, vestibular neuritis, viral labyrinthitis and Ménière's disease showed a consistent and distinct vestibular deficit in one ear. Patients with migraine, Chiari malformations, general viral infections, genetic disorders, degenerative disorders and diabetes revealed a diffuse pattern, i.e. vestibular deficits were detected in both ears and possibly suggesting an underlying common cause. Following electrocochleography the electrode can remain in situ for octave analysis of the peripheral vestibular organ allowing a functional assessment of the whole inner ear.

It is expected that other sequences of octaves and their relationship to each other can accurately describe vestibular function or dysfunction. The invention defines a principle that may be applicable to other sensory modalities.

Whilst the above description includes the preferred embodiments of the invention, it is to be understood that many variations, alterations, modifications and/or additions may be introduced into the constructions and arrangements of parts previously described without departing from the essential features or the spirit or ambit of the invention.

It will be also understood that where the word “comprise”, and variations such as “comprises” and “comprising”, are used in this specification, unless the context requires otherwise such use is intended to imply the inclusion of a stated feature or features but is not to be taken as excluding the presence of other feature or features.

The reference to any prior art in this specification is not, and should not be taken as, an acknowledgment or any form of suggestion that such prior art forms part of the common general knowledge.

TABLE 1

| | | | Reference | Octave | Octave | | Reference | Octave | Octave | |
|---|---|---|---|---|---|---|---|---|---|---|
| | | | Frequency | Ratio | Ratio | Deficit % | Frequency | Ratio | Ratio | Deficit % |
| Subject | Diagnosis | Left/Right | Roll Hz | ipsi | contra | (Jongkees) | Pitch Hz | fward | bward | (Jongkees) |
| L. S. | Norm | Left | 24.4 | 17.9 | 28.5 | 30.5 | 23.3 | 1.4 | 74 | |
| | | Right | 21.7 | 3.1 | 21.6 | | 23.1 | 0.5 | 111.9 | 19.7 |
| T. F. | Norm | Left | 23 | 1.8 | 40.4 | 35.9 | 23.9 | 0 | 45.8 | |
| | | Right | 23.6 | 0.7 | 19.2 | | 24.9 | 4.7 | 99.2 | 38.8 |
| F. A. | Norm | Left | 21.1 | 2 | 135.1 | 14.5 | 21.3 | 0 | 67.5 | 14.3 |
| | | Right | 21.2 | 2.4 | 100 | | 24.4 | 1 | 49.6 | |
| B. K. F. | Norm | Left | 24.2 | 5 | 32.1 | | 24.5 | 2.1 | 11.2 | |
| | | Right | 23.1 | 1 | 39 | 3.8 | 23 | 2.1 | 27.5 | 38 |
| L. K. | Norm | Left | 24 | 1.3 | 140 | 13.4 | 24.5 | 1.3 | 57.4 | |
| | | Right | 24.9 | 3.3 | 104.6 | | 23.1 | 3.3 | 136.7 | 40.9 |
| Average | | | | | | 19.62 | | | | 3.34 |
| Stdev | | | | | | 13.2154 | | | | 12.3718 |
| 2 Stdev | | | | | | 26.4308 | | | | 24.7437 |
| Range | | | | | | <46% | | | | <55% |

Table 1 is shows octave analysis in roll and in pitch in normal subjects. Normal responses in roll are expected to be below 46% in roll and below 55% in pitch.

TABLE 2

| | | | Reference | | | |
|---|---|---|---|---|---|---|
| | | | Frequency | Octave | Octave | |
| | Diag- | | Cold-Base | Ratio | Ratio | Deficit % |
| Subject | nosis | Left/Right | Hz | Cold | Warm | (Jongkees) |
| L. S. | Norm | Left | 24.1 | 111.1 | 77.4 | 27.4 |
| | | Right | 22.2 | 92.7 | 14.7 | |
| T. F. | Norm | Left | 21.1 | 22.6 | 111.1 | |
| | | Right | 24.3 | 118.9 | 45.1 | 10.2 |
| F. A. | Norm | Left | 22.4 | 65.4 | 165.4 | 11.6 |
| | | Right | 22.6 | 49.5 | 133.2 | |
| B. K. F. | Norm | Left | 21.4 | 113.3 | 4.9 | 2.6 |
| | | Right | 22.6 | 30.4 | 94.1 | |
| L. K. | Norm | Left | 24.2 | 178.9 | 95.5 | 19.1 |
| | | Right | 24.4 | 3.8 | 182.7 | |
| Average | | | | | | 14.18 |
| Stdev | | | | | | 9.4282 |
| 2 Stdev | | | | | | 18.6565 |
| Range | | | | | | <33% |

Cold and warm stimuli were referenced to the cold baseline recording with the patient lying on a bench and the head elevated by 30°. The table shows figures of five normal subjects. Normal responses are expected to be below 33%.

### REFERENCES

1. Gibson W P R. Essentials of clinical electric response audiometry. Churchill Livingstone, Edinburgh London and New York 1978
2. Franz B. Electro-Otolithography: New insight into Benign Paroxysmal Positioning Vertigo. Int Tinnitus J 9: 92-96, 2003
3. Unterberger S. Neue objektiv registrierbare Vestibularis-Körperdreh-Reaktionen, erhalten durch Treten auf der Stelle. Arch klin exp Ohr-, Nas, -u Kehlk-Heilk 145: 478-, 1938
4. Dix M, Hallpike C S. The pathology-symptomatology and diagnosis of certain common disorders of the vestibular system. Proc roy Soc Med, Sect Otol 45, 341-354, 1952
5. Halmagyi G M, Curthoys I S. A clinical sign of canal paresis. Arch Neurol 45: 737-739, 1988
6. Romberg M H. Lehrbuch der Nervenkrankheiten des Menschen. Duncker, Berlin 1846
7. Ashan G, Bergstedt M, Stahle J. Nystagmography. Recording of nystagmus in clinical neuro-otological examination. Acta Otolaryngol Suppl 129: 1-103, 1956
8. Colebatch J G, Halmagyi G M, Skuse N F. Myogenic potentials generated by click evoked vestibulocollic reflex. J Neurol Neurosurg and Psych 57: 190-197, 1994
9. Claussen C F. Objective and quantitative vestibular spinal testing by means of the Computer-Video-Cranio-Corpo-Graphy (CVCCG). Advances in ORL, 42: 43-49, Karger, Basel 1988
10. Jongkees L B W. Physiologie und Untersuchungsmethoden des Vestibularsystems. Hals-Nasen-Ohrenheilkunde in Praxis und Klinik, Chapter 16. Editors Berendes Link Zöllner, Georg Thieme 1979
11. Charlet de Sauvage R, Dolivet G, Erre J P, Aran J M. Electrovestibulography in experimental animals. Physiologist 33 Suppl: 117-118, 1990
12. Plotnik M, Mager M, Elidan J, Sohmer H. Short latency vestibular evoked potentials (VsEPs) to linear acceleration impulses in rats. Electroencephalogr Clin Neurophysiol 104: 522-530, 1997
13. Charlet de Sauvage R, Erre J P, Aran J M. Differential sensitivity to rotation measured on potentials evoked by electrical stimulation of the guinea pig ear. Electroencephalogr Clin Neurophysiol 92: 462-468, 1994
14. Fernandez C, Goldberg J M. Physiology of peripheral neurons innervating otolith organs of the squirrel monkey. I. Response to static tilts and to long-duration centrifugal force. J Neurophysiol 39: 970-984, 1976

1. A method of examining a bodily function comprising:
detecting a first set of frequency characteristics and amplitude characteristics of a first electrical signal deriving from rhythmic activity of a nerve in a living body at a first condition;
stimulating said nerve and detecting a second set of frequency characteristics and amplitude characteristics of a second electrical signal deriving from the rhythmic activity of said nerve in a stimulated second condition;
identifying the fundamental frequency present in said frequency characteristics;
calculating from said fundamental frequency the frequencies corresponding to a plurality of octaves of said fundamental frequency;
identifying the amplitude of said electrical signals at said octaves;
calculating for said first signal a first amplitude ratio being the ratio of:
the amplitude difference between a first pair of said octaves, and
the amplitude difference between a second pair of said octaves;
calculating for said second signal a second amplitude ratio being the ratio of:
the amplitude difference between said first pair of octaves, and
the amplitude difference between said second pair of octaves;
calculating an octave ratio being the ratio of said first amplitude ratio to said second amplitude ratio; and
using said octave ratio as a measure of said bodily function.

2. A method of diagnosing a bodily dysfunction, said method comprising:
detecting a first set of frequency characteristics and amplitude characteristics of a first electrical signal deriving from rhythmic activity of the peripheral vestibular nerve in a living body at a first condition;
stimulating said nerve and detecting a second set of frequency characteristics and amplitude characteristics of a second electrical signal deriving from the rhythmic activity of said nerve in a stimulated second condition;
identifying the fundamental frequency present in said frequency characteristics;
calculating from said fundamental frequency the frequencies corresponding to a plurality of octaves of said fundamental frequency;
identifying the amplitude of said electrical signals at said octaves;
calculating for said first signal a first amplitude ratio being the ratio of:
the amplitude difference between a first pair of said octaves, and
the amplitude difference between a second pair of said octaves;
calculating for said second signal a second amplitude ratio being the ratio of:
the amplitude difference between said first pair of octaves, and
the amplitude difference between said second pair of octaves;
calculating an octave ratio being the ratio of said first amplitude ratio to said second amplitude ratio; and
using said octave ratio as a measure of said bodily dysfunction.

3-5. (canceled)

6. A method according to claim 1 wherein said first pair of octaves and said second pair of octaves are selected from octaves 1 to 7 of said fundamental frequency.

7. A method according to claim 6 wherein said first pair of octaves and said second pair of octaves are selected from octaves 2 to 4 of said fundamental frequency.

8. A method according to claim 1 wherein said first amplitude ratio is the ratio of:
the amplitude difference between octave 2 and octave 3 of said fundamental frequency, and
the amplitude difference between octave 2 and octave 4 of said fundamental frequency.

9. A method according to claim 6 wherein said first amplitude ratio is the ratio of:
the amplitude difference between octave 2 and octave 3 of said fundamental frequency, and
the amplitude difference between octave 2 and octave 4 of said fundamental frequency.

10. A method according to claim 7 wherein said first amplitude ratio is the ratio of:
the amplitude difference between octave 2 and octave 3 of said fundamental frequency, and
the amplitude difference between octave 2 and octave 4 of said fundamental frequency.

11. A method according to claim 2 wherein said first pair of octaves and said second pair of octaves are selected from octaves 1 to 7 of said fundamental frequency.

12. A method according to claim 11 wherein said first pair of octaves and said second pair of octaves are selected from octaves 2 to 4 of said fundamental frequency.

13. A method according to claim 2 wherein said first amplitude ratio is the ratio of:
the amplitude difference between octave 2 and octave 3 of said fundamental frequency, and
the amplitude difference between octave 2 and octave 4 of said fundamental frequency.

14. A method according to claim 11 wherein said first amplitude ratio is the ratio of:
the amplitude difference between octave 2 and octave 3 of said fundamental frequency, and
the amplitude difference between octave 2 and octave 4 of said fundamental frequency.

15. A method according to claim 12 wherein said first amplitude ratio is the ratio of:
the amplitude difference between octave 2 and octave 3 of said fundamental frequency, and
the amplitude difference between octave 2 and octave 4 of said fundamental frequency.
